(19) United States
(12) Patent Application Publication
(10) Pub. No.: US 2007/0100331 A1
(43) Pub. Date: May 3, 2007

(54) Systems and methods for organ tissue ablation

(75) Inventors: Kimbolt Young, Newtonville, MA (US); Kathleen Fernald, Brookline, MA (US)

Correspondence Address: Vista IP Law Group LLP, 2040 MAIN STREET, 9TH FLOOR, IRVINE, CA, 92614, US

(73) Assignee: Boston Scientific Scimed, Inc.

(21) Appl. No.: 11/261,211

(22) Filed: Oct. 27, 2005

(51) Int. Cl. A61B 18/14

(52) U.S. Cl. 606/41

(57) ABSTRACT

A system for treating organ tissue includes a source of electrical energy, a first electrode coupled to the energy source, the first electrode having a surface configured for electrically coupling with a surface of an organ, and a second electrode coupled to the energy source, the second electrode having a tissue-piercing distal tip configured for piercing the organ such that the second electrode electrically couples with internal tissue of the organ. A system for treating organ tissue includes a source of electrical energy, a first electrode coupled to the energy source and having a surface configured for electrically coupling with a surface of an organ at a first position, and a second electrode coupled to the energy source and having a surface configured for electrically coupling with the surface of the organ at a second position.

### BACKGROUND

1. Field

The field of the application relates to medical devices, and more particularly, to systems and methods for ablating or otherwise treating tissue using electrical energy.

2. Background

Tissue may be destroyed, ablated, or otherwise treated using thermal energy during various therapeutic procedures. Many forms of thermal energy may be imparted to tissue, such as radio frequency electrical energy, microwave electromagnetic energy, laser energy, acoustic energy, or thermal conduction.

In particular, radio frequency ablation (RFA) may be used to treat patients with tissue anomalies, such as liver anomalies and many primary cancers, such as cancers of the stomach, bowel, pancreas, kidney and lung. RFA treatment involves the destroying undesirable cells by generating heat through agitation caused by the application of alternating electrical current (radio frequency energy) through the tissue.

Various RF ablation devices have been suggested for this purpose. For example, U.S. Pat. No. 5,855,576 describes an ablation apparatus that includes a plurality of wire electrodes deployable from a cannula or catheter. Each of the wires includes a proximal end that is coupled to a generator, and a distal end that may project from a distal end of the cannula. The wires are arranged in an array with the distal ends located generally radially and uniformly spaced apart from the catheter distal end. The wires may be energized in a monopolar or bipolar configuration to heat and necrose tissue within a precisely defined volumetric region of target tissue. The current may flow between closely spaced wire electrodes (bipolar mode) or between one or more wire electrodes and a larger, common electrode (monopolar mode) located remotely from the tissue to be heated.

Generally, ablation therapy uses heat to kill tissue at a target site. The effective rate of tissue ablation is highly dependent on how much of the target tissue is heated to a therapeutic level. In certain situations, complete ablation of target tissue that is adjacent a vessel may be difficult or impossible to perform, since significant bloodflow may draw the produced heat away from the vessel wall, resulting in incomplete necrosis of the tissue surrounding the vessel. This phenomenon, which causes the tissue with greater blood flow to be heated less, and the tissue with lesser blood flow to be heated more, is known as the “heat sink” effect. It is believed that the heat sink effect is more pronounced for ablation of tissue adjacent large vessels that are more than 3 millimeters (mm) in diameter. Due to the increased vascularity of the liver, the heat sink effect may cause recurrence of liver tumors after a radio frequency ablation.

Also, because of the vascularity of the liver, resection of a portion of a liver (as is required by some surgeries) may result in significant bleeding. Existing techniques in managing bleeding of a resected liver include delivering embolic material within a vessel of a liver to prevent blood flow. However, such technique is time consuming, may require complex imaging modality, and may not be effective in the case in which a relatively large portion of a liver is being resected.

### SUMMARY

In accordance with some embodiments, a system for treating organ tissue includes a source of electrical energy, a first electrode coupled to the energy source, the first electrode having a surface configured for electrically coupling with a surface of an organ, and a second electrode coupled to the energy source, the second electrode having a tissue-piercing distal tip configured for piercing the organ such that the second electrode electrically couples with internal tissue of the organ.

In accordance with other embodiments, a method of performing an organ tissue ablation procedure includes placing a first electrode at a first position on a surface of an organ, piercing the organ with a second electrode to position the second electrode inside the organ, and applying electrical energy through a circuit formed by the first and second electrodes to ablate a portion of the organ.

In accordance with other embodiments, a system for treating organ tissue includes a source of electrical energy, a first electrode coupled to the energy source and having a surface configured for electrically coupling with a surface of an organ at a first position, and a second electrode coupled to the energy source and having a surface configured for electrically coupling with the surface of the organ at a second position.

In accordance with other embodiments, a method of performing a liver ablation procedure includes placing a first electrode at a first position on a surface of a liver, placing a second electrode at a second position on the surface the liver, and applying electrical energy through an electrical circuit formed by the first and the second electrodes to ablate a portion of the liver.

Other aspects and features of the embodiments will be evident from reading the following description of the embodiments.

### BRIEF DESCRIPTION OF THE DRAWINGS

The drawings illustrate the design and utility of embodiments of the application, in which similar elements are referred to by common reference numerals. In order to better appreciate how advantages and objects of various embodiments are obtained, a more particular description of the embodiments are illustrated in the accompanying drawings. Understanding that these drawings depict only typical embodiments of the application and are not therefore to be considered limiting its scope, the embodiments will be described and explained with additional specificity and detail through the use of the accompanying drawings.

FIG. 1 illustrates an ablation system for treating tissue in accordance with some embodiments;

FIG. 2 illustrates a method of using the ablation system of FIG. 1 in accordance with some embodiments;

FIG. 3 illustrates an ablation system for treating tissue in accordance with other embodiments;

FIG. 4 illustrates a variation of the ablation system of FIG. 3 in accordance with some embodiments;

FIG. 5 illustrates a method of using the ablation system of FIG. 4 in accordance with some embodiments;

FIG. 6 illustrates a method of using the ablation system of FIG. 4 in accordance with other embodiments;

FIG. 7 illustrates the ablation system of FIG. 4, showing the ablation system further having a securing device for securing electrodes against a tissue surface;

FIG. 8 illustrates an ablation system for treating tissue in accordance with other embodiments, showing the ablation system having an electrode with an envelope configuration;

FIG. 9 illustrates an ablation system for treating tissue in accordance with other embodiments, showing the ablation system having two electrodes each of which having a surface for contacting an organ surface;

FIG. 10 illustrates a method of using the ablation system of FIG. 9 in accordance with some embodiments; and

FIG. 11 illustrates a variation of the ablation system of FIG. 9 in accordance with some embodiments.

### DESCRIPTION OF THE EMBODIMENTS

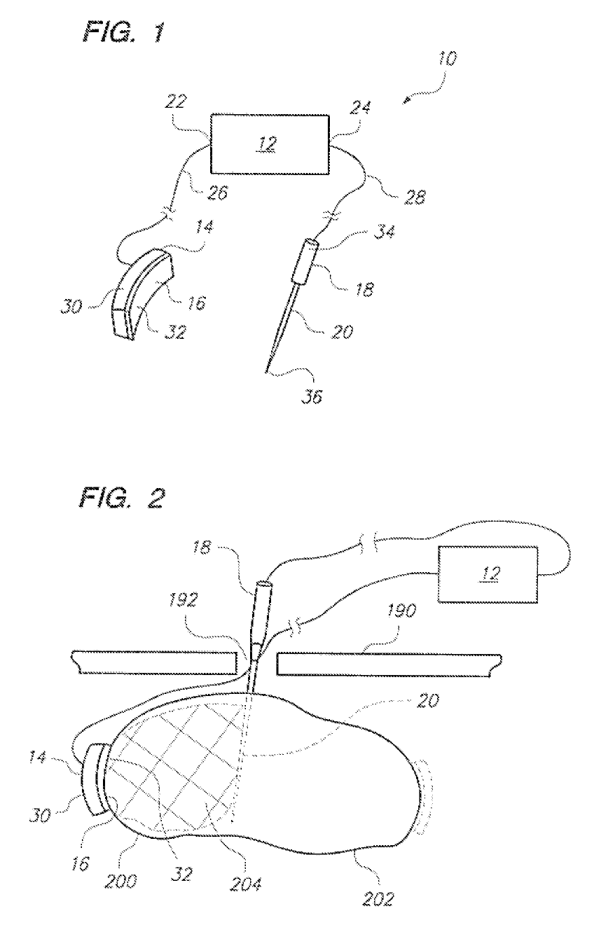

FIG. 1 illustrates an ablation system 10 in accordance with some embodiments. The ablation system 10 includes a source of energy 12, e.g., a radio frequency (RF) generator, a first device 14 carrying a first electrode 16, and a second device 18 carrying a second electrode 20. The source of energy 12 has a first terminal 22 and a second terminal 24. The ablation system 10 further includes a first cable 26 for electrically coupling the first electrode 16 to the first terminal 22, and a second cable 28 for electrically coupling the second electrode 20 to the second terminal 24.

The generator 12 is preferably capable of operating with a fixed or controlled voltage so that power and current diminish as impedance of the tissue being ablated increases. Exemplary generators are described in U.S. Pat. No. 6,080,149, the disclosure of which is expressly incorporated by reference herein. The preferred generator 12 may operate at relatively low fixed voltages, typically below one hundred fifty volts (150 V) peak-to-peak, and preferably between about fifty and one hundred volts (50-100 V). Such radio frequency generators are available from Boston Scientific Corporation, assignee of the present application, as well as from other commercial suppliers. It should be noted that the generator 12 is not limited to those that operate at the range of voltages discussed previously, and that generators capable of operating at other ranges of voltages may also be used.

In the illustrated embodiments, the first device 14 has a structure 30 that is made from a flexible material, such as an elastic metal or a polymer. The first electrode 16, which is also made from an elastic material (e.g., a bendable metal), is secured to the structure 30, and has a surface 32 for contacting tissue, such as a surface of an organ. In some embodiments, the structure 30 is capable of being bent from a first configuration to a second configuration via a force, and is capable of remaining in the second configuration upon a removal of the force. Such feature allows a desired profile of the surface 32 to be created during use. Alternatively, the structure 30 and/or the first electrode 16 can be made from a rigid material that prevents the first electrode 16 from being bent. As shown in the figure, the surface 32 of the first electrode 16 has a planar configuration. As used in this specification, the term “planar configuration” refers to a configuration that can have a two dimensional characteristic (as that of a perfectly flat plane), or a three dimensional characteristic (as that of a surface having one or more portions that do not lie in a perfectly flat plane).

In other embodiments, the first device 14 can have other configurations. For example, in other embodiments, the first device 14 can further include a handle secured to the structure 30, which allows a physician to press the electrode surface 32 towards a tissue surface. In further embodiments, the first device 14 can include an elongate shaft connected between the handle and the structure 30. The shaft can be elastic (which allows a physician to bent the shaft into a desired profile during use), or rigid. During use, the elongate shaft allows a physician to reach tissue with the first electrode 16.

The second device 18 includes a handle 34 to which the second electrode 20 is secured. The second electrode 20 has a rectilinear profile, but alternatively, can have a curvilinear profile, or any of other non-linear profiles. As shown in the figure, the second electrode 20 also has a sharp distal tip 36 for piercing tissue. In other embodiments, the second device 18 can have other configurations. For example, in other embodiments, the second device 18 can include a cannula having a lumen. In such cases, the second electrode 36 can include one or more tines that assume a low profile when confined within the lumen of the cannula, and assume a relaxed and expanded profile when unconfined outside the lumen of the cannula. Examples of such device are described in U.S. Pat. No. 5,855,576, the entire disclosure of which is expressly incorporated by reference herein.

FIG. 2 illustrates a method of ablating tissue using the ablation system 10 of FIG. 1 in accordance with some embodiments. First, an incision is made on a patient's skin 190 to create an opening 192. The first device 14 is then inserted through the opening 192 (percutaneously) and the first electrode 16 is placed against a surface 200 of an organ 202 (e.g., a liver). In some embodiments, the first electrode 16 can be secured to the surface 200 using one or more hooks coupled to the electrode 16 (e.g., at the periphery of the electrode 16). In such cases, the hook(s) penetrate within the tissue to thereby secure the electrode 16 relative to the surface 200. Alternatively, a suction device located next to the electrode 16 (e.g., at a periphery of the electrode 16) can be used to secure the electrode 16 relative to the surface 200. In such cases, the suction device creates a suction, and pulls the organ surface 200 towards the electrode 16, thereby stabilizing the electrode 16 relative to the surface 200. Other methods of securing the electrode 16 relative to the surface 200 can also be used. If the first device 14 includes a handle and a shaft, these components can be used as leverage to press the first electrode 16 against the surface 200. The second device 18 is then inserted through the opening 192, and the second electrode 20 pierces into the organ 202 using the distal tip 36. Alternatively, the second device 18 can be inserted through the opening 192 before the first device 14.

In alternative embodiments, one or more components or elements may be provided for introducing the devices 14, 18 through the patient's skin 190. For example, a conventional sheath (not shown) may be inserted through the patient's skin 190 to gain access to the organ 202. Once properly positioned, the first and second devices 14, 18 may then be introduced through the sheath lumen to reach the organ 202.

In some embodiments, before the first device 14 is inserted into the patient, if the structure 30 of the first device 14 is flexible, a physician can bend the structure 30 to thereby form the electrode surface 16 into a desired profile (bent configuration). For example, the electrode surface 16 can be bent such that its profile resembles a contour of a target surface of the organ 202 at which the electrode 16 will be placed.

Next, energy, preferably RF electrical energy, may be delivered from the generator 12 to the first electrode 16, with the second electrode 20 functioning as a return electrode, thereby creating a lesion 204 between the first and second electrodes 16, 20. Alternatively, the generator 12 may deliver energy to the second electrode 20, with the first electrode 16 functioning as a return electrode. In some embodiments, after the lesion 204 has been created, the ablation system 10 (or another ablation device/system) can be used to ablate a target treatment site (e.g., a tumor) located on one side of the lesion 204. In such cases, the formed lesion 204 can be used as a barrier to prevent blood from flowing from one side of the lesion 204 to the other side of the lesion 204, thereby allowing the target treatment site located on one side of the lesion 204 to be ablated efficiently without being affected by a heat sink effect due to blood flow.

In some cases, if it is desired to perform further ablation to increase the lesion size or to create additional lesion(s) at different site(s) of the organ 202, one or both of the first electrode 16 and the second electrode 20 may be positioned, and be placed at different location(s), and the same steps discussed previously may be repeated. For example, in some embodiments, after the first lesion has been created, the first electrode 16 may be placed on the other side of the organ 204 (indicated by dotted lines), with the second electrode 20 remaining in its first position. The electrodes 16, 20 can then be used to create a second lesion, thereby forming an ablation plane substantially across an entire cross section of the organ 202 with the first lesion. In some cases, after a lesion across a substantial cross section of the organ 202 has been created, part of the organ 202 on one side of the ablation plane can be surgically removed (resect).

In the above embodiments, the first and second electrodes 16, 20 are used to create the lesion 204 in a bipolar configuration. Alternatively, the lesion 204 can be created in a monopolar configuration. In such cases, the first and the second electrodes 16, 20 may be connected to the active terminal 22 of the generator 12 using a “Y” cable, and a common ground pad electrode (not shown) is electrically coupled to the terminal 24. The first and second electrodes 16, 20 then deliver energy to the common ground pad electrode, which is generally placed on a patient's skin, in a monopolar mode.

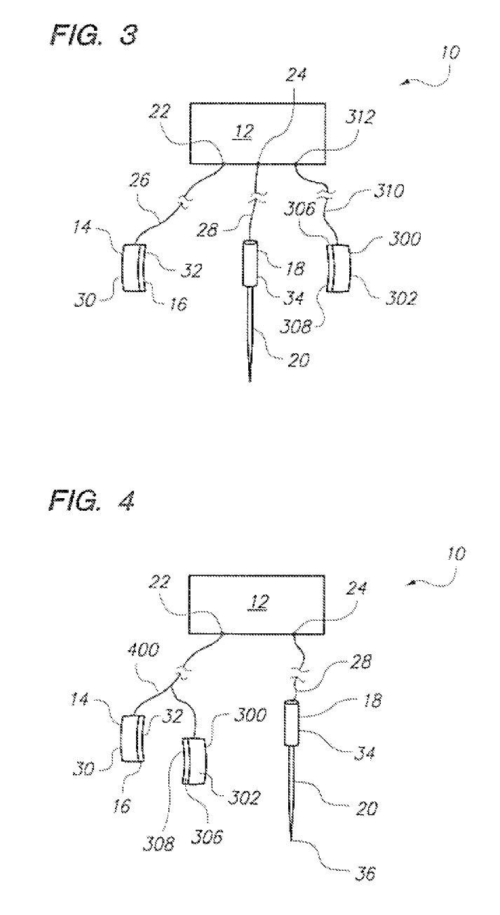

FIG. 3 illustrates an ablation system 10 in accordance with other embodiments. The ablation system 10 is the same as that described with reference to FIG. 1, except that the ablation system 10 of FIG. 3 further includes a third device 300 having a structure 302 for carrying a third electrode 306. Similar to the first electrode 16, the third electrode 306 has a surface 308 for contacting tissue surface (e.g., surface of an organ). The ablation system 10 further includes a third cable 310 that electrically couples the third electrode 306 to a third terminal 312 on the source of energy 12. The output terminals 22, 312 of the generator 12 may be coupled to common control circuits (not shown) within the generator 12. Alternatively, the generator 12 may include separate control circuits coupled to each of the output terminals 22, 312. The control circuits may be connected in parallel with one another, yet may include separate impedance feedback to control energy delivery to the respective output terminals 22, 312. In some embodiments, the output terminals 22, 312 may be connected in parallel to an active terminal of the generator 12 such that the first and third electrodes 16, 306 can deliver energy to a common ground pad electrode (not shown) in a monopolar mode, or to the second electrode 20 in a bipolar mode. Alternatively, the output terminals 22, 312 may be connected to opposite terminals of the generator 12 for delivering energy between the first and third electrodes 22, 312 in a bipolar mode.

In further embodiments, the generator 12 does not have the third terminal 312. Instead, the first and the third electrodes 16, 306 are electrically coupled to each other via a cable. In such cases, the cable is electrically coupled to the first terminal, which supplies electrical energy to the first and the third electrodes 16, 306. The first and the third electrodes 16, 306 form a first pole of a circuit, and the second electrode 20 form a second pole of the circuit.

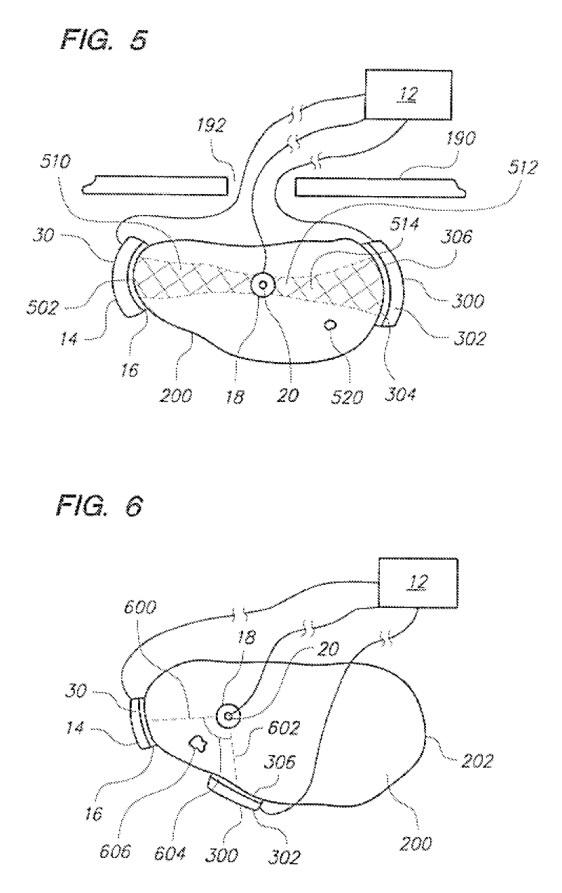

In other embodiments, if the source of energy 12 has only two terminals 22, 24, a “Y” cable 400 can be provided to electrically couple the first and third electrodes 16, 306 to the first terminal 22 (FIG. 4).

FIG. 5 illustrates a method of ablating tissue using the ablation system 10 of FIG. 4 in accordance with some embodiments. First, an incision is made on a patient's skin 190 to create an opening 192. The first device 14 is then inserted through the opening 192 (percutaneously) and the first electrode 16 is placed at a first location 502 against a surface 200 of an organ 202 (e.g., a liver). The second device 18 is then inserted through the opening 192, and the second electrode 20 pierces into the organ 202 using the distal tip 36. The third device 300 is then inserted through the opening 192 and the third electrode 306 is placed at a second location 504 against the surface 200 of the organ 202. Alternatively, the order of inserting the first, second, and third devices 14, 18, 300 can be different from that described previously. In the illustrated embodiments, the first, second, and third electrodes 16, 20, 306 are positioned such that they lie approximately within a flat (or linear) plane.

In alternative embodiments, one or more components or elements may be provided for introducing the devices 14, 18, 300 through the patient's skin 190. For example, a conventional sheath (not shown) may be inserted through the patient's skin 190 to gain access to the organ 202. Once properly positioned, the first, second, and third devices 14, 18, 300 may then be introduced through the sheath lumen to reach the organ 202.

In some embodiments, before the first device 14 is inserted into the patient, if the structure 30 of the first device 14 is flexible, a physician can bend the structure 30 to thereby form the electrode surface 16 into a desired profile (bent configuration). For example, the electrode surface 16 can be bent such that its profile resembles a contour of a portion of the surface 200 (e.g., the surface portion at the first location 502) at which the first electrode 16 will be placed. Similarly, before the third device 300 is inserted into the patient, if the structure 302 of the third device 300 is flexible, a physician can bend the structure 302 to thereby form the electrode surface 308 into a desired profile (bent configuration). For example, the electrode surface 308 can be bent such that its profile resembles a contour of a portion of the surface 200 (e.g., the surface portion at the second location 504) at which the third electrode 306 will be placed.

Next, energy, preferably RF electrical energy, may be delivered from the generator 12 to the first and third electrodes 16, 306, with the second electrode 20 functioning as a return electrode, thereby creating a first lesion 510 between the first and second electrodes 16, 20, and a second lesion 512 between the second and third electrodes 20, 306. Alternatively, the generator 12 may deliver energy to the second electrode 20, with the first and third electrodes 16, 306 functioning as return electrodes. In some embodiments, after the lesion 514 has been created, the ablation system 10 (or another ablation device/system) can be used to ablate tissue at a target treatment site 520 (e.g., a tumor) located on one side of the lesion 514. In such cases, the formed aggregate lesion 514 (formed by lesions 510, 512) can be used as a barrier to prevent blood from flowing from one side of the lesion 514 to the other side of the lesion 514, thereby allowing the target treatment site 520 located on one side of the lesion 514 to be ablated efficiently without being affected by a heat sink effect due to blood flow.

In some embodiments, if the first and third electrodes 16, 306 are sufficiently large, the above technique will result in an ablation plane formed substantially across an entire cross section of the organ 202. Alternatively, if the first and third electrodes 16, 306 are not sufficiently large, one or both of the first and third electrodes 16, 306 can be positioned, and the above technique is repeated until a lesion substantially across an entire cross section of the organ 202 is formed. In some cases, after a lesion across a substantial cross section of the organ 202 has been created, part of the organ 202 on one side of the ablation plane can be surgically removed, e.g., by cutting through the ablated region. The ablated region acts as a shield to prevent, or at least reduce, bleeding after the resection of the organ 202.

FIG. 6 illustrates another method of ablating tissue using the ablation system 10 of FIG. 4 in accordance with other embodiments. As shown in the figure, the first, second, and third electrodes 18, 20, 306 are positioned relative to each other such that a first line 600 extending between the first electrode 16 and the second electrode 20, and a second line 602 extending between the second electrode 20 and the third electrode 306, form an non-180° angle. In some embodiments, such arrangement of the electrodes 18, 20, 36 can be used to perform a wedge resection in which a first resection (or ablation) plane is created between the first and second electrodes 18, 20, and a second resection (or ablation) plane is created between the second and third electrodes 20, 306, thereby resecting tissue that contains a tumor 606.

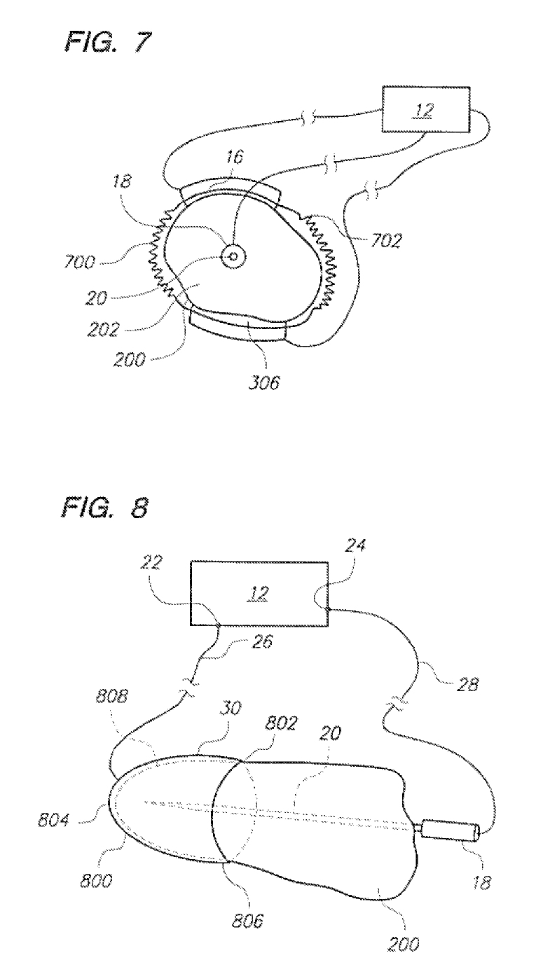

In the above embodiments, the first electrode 16 (and the third electrode 306) are secured to tissue surface by a physician applying a force to press the electrode 16 (and electrode 306) against the tissue surface. In other embodiments, any of the ablation systems 10 described herein can further include a securing device for securing the first electrode 16 and the third electrode 306 against tissue surface (e.g., surface of an organ). FIG. 7 illustrates the ablation system 10 of FIG. 4, which further includes two elastic bands 700, 702 for securing the first electrode 16 and the third electrode 306 against the surface 200 of the organ 202. The elastic bands 700, 702 can be a rubber band, a spring, or any of other elastic structures (including structures made from nylon, elastic polymers, or any of other elastic materials). During use, the first electrode 16 and the third electrode 306 are placed at different locations along the surface 200 of the organ 202, with the elastic bands 700, 702 wrapped at least partially around parts of the organ 202. The elastic bands 700, 702 pull the first and the third electrodes 16, 306 towards each other, thereby applying a compression force to push the first electrode 16 and the third electrode 306 towards the surface 200.

In other embodiments, the ablation system 10 can include other types of securing devices for securing the first electrode 16 (and the third electrode 306) against a tissue surface. For example, in other embodiments, the ablation system 10 can further include a suction device (not shown), and a tube (not shown) having a first end connected to the suction device, and a second end connected to the first device 14. In some embodiments, the second end of the tube can be located adjacent to the first electrode 16. In other embodiments, the first electrode 16 can include an opening, which is in fluid communication with the lumen of the tube. During use, the suction device applies a suction through the tube, thereby pulling a tissue surface towards the first electrode 16 to secure the first electrode 16 relative to the tissue surface.

FIG. 8 illustrates a variation of the ablation system 10 in accordance with other embodiments. The ablation system 10 is similar to that described with reference to FIG. 1, except that the structure 30 of the first device 14 is an envelope 800 having an opening 802 at one end, and a lumen 808 for accommodating a portion of the organ 202. In some embodiments, the envelope 800 itself is made from a conductive material, thereby allowing the structure 30 to function as the electrode 16. For example, the envelope 800 can be made from a plurality of metallic wires/strands that are weaved into a sock-like structure. In other embodiments, the structure 30 can be made from a non-conductive material. In such cases, at least part of the structure 30 can be covered with a conductive material (e.g., strands of metallic wires, metallic particles, or conductive pads) to form the electrode 16. In the illustrated embodiments, the envelope 800 has a closed end 804. In other embodiments, the structure 30 can have an opening at the end 804, and resembles a tube or a ring.

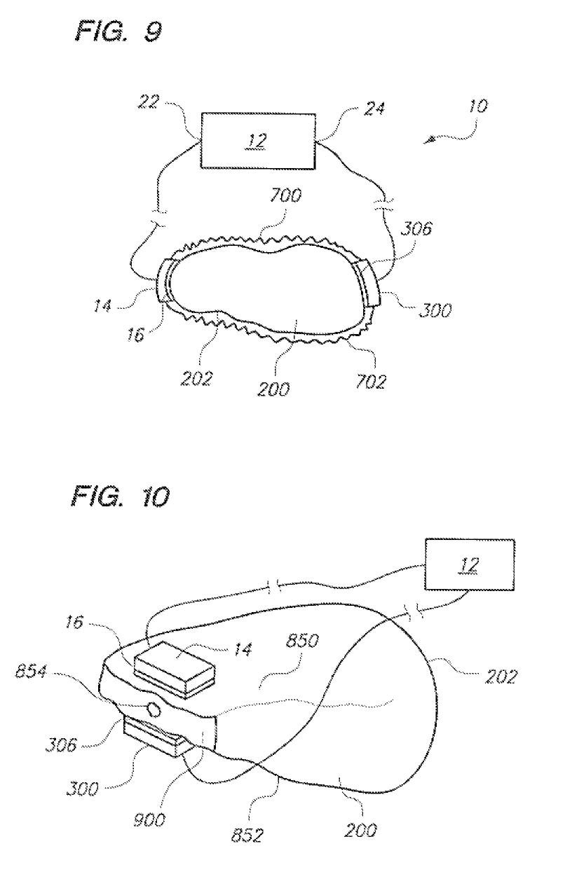

FIG. 9 illustrates a variation of the ablation system 10 of FIG. 4 in accordance with other embodiments. The ablation system 10 is similar to that described with reference to FIG. 4, except that it does not include the second device 18 and the second electrode 20. In such cases, the first electrode 16 is electrically coupled to the first terminal 22 of the energy source 12, and the third electrode 306 is electrically coupled to the second terminal 24 of the energy source 12. During use, the electrodes 16, 306 are used to ablate tissue in a bipolar configuration.

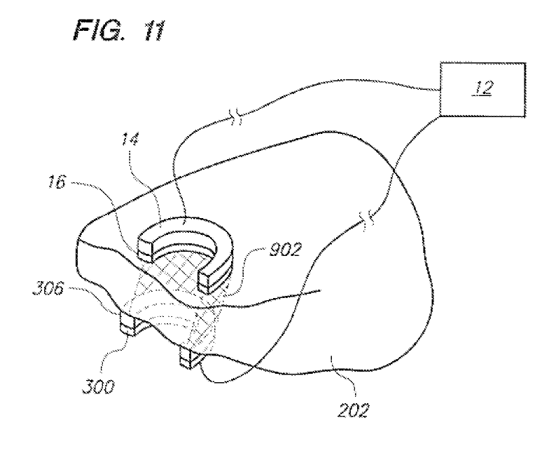

In some embodiments, the ablation system 10 of FIG. 9 can be used to create a lesion (a transmural lesion) across a thickness of an organ. As shown in FIG. 10, the first electrode 16 can be placed on one side 850 of the organ 202, with the third electrode 306 placed on the opposite side 852 of the organ 202. The first and the third electrodes 16, 306 can then be used to deliver ablation energy to ablate tissue 900 between the electrodes 16, 306 (e.g., to ablate a tumor 854).

In any of the embodiments described herein, the structure 30 and the electrode 16 can be made from a material, and have respective thicknesses that are thin enough, such that a physician can cut (e.g., using a scissor, a knife, or any of other known cutting devices) the structure 30 and the electrode 16 into a desired shape during use. For example, in some embodiments, the structure 30 can be made from a polymer, and has a thickness that is less than 10 millimeters (mm). Also, in some embodiments, the electrode 16 can include a substrate made from a material (e.g., a polymer) that can be cut, with at least a portion of the substrate covered by a conductive material. In other embodiments, the electrode 16 can be made from a metal that can be cut. For example, in some embodiments, the electrode 16 can be a foil. FIG. 11 illustrates an embodiments of the ablation system 10 of FIG. 9, with the first electrode 16 and the third electrode 306 each cut into a “C” shape. During use, the first and the third electrodes 16, 306 are placed on different sides of the organ 202, and a “C” shape ablation plane 902 can be created between the first and the third electrodes 16, 306. In other embodiments, each of the first and the third electrodes 16, 306 can be cut into other shapes, such as a “V” shape or an “O” shape.

It should be noted that the ablation system 10 is not necessarily limited to the configurations described previously, and that the ablation system 10 can have other configurations in other embodiments. For example, in other embodiments, the first electrode 16 and the third electrode 306 can have different shapes and/or sizes. Also, in other embodiments, instead of having the electrodes 16, 20, 306, for delivering RF energy, the ablation system 10 can include other types of ablation devices. For example, in other embodiments, the ablation system 10 can include ablation devices connected to the energy source 12, wherein each of the ablation devices is configured for delivering other form of energy, such as ultrasound energy, or microwave energy, for the purpose of ablation.

Also, instead of delivering ablation energy in a bipolar configuration, any of the embodiments of the ablation systems 10 described herein can be modified to allow delivery of ablation energy in a monopolar configuration. For example, in the embodiments of FIG. 9, the first and third electrodes 16, 306 can be electrically coupled to the first terminal 22 using a “Y” cable, and a neutral or ground electrode (e.g., an external electrode pad) may be electrically coupled to the opposite terminal 24 of the generator 12. In such cases, the ground electrode can be coupled to the patient, e.g., be placed on the patient's skin, and the electrodes 16, 306 can then be used to deliver ablation energy in a monopolar configuration.

Thus, although several embodiments have been shown and described, it would be apparent to those skilled in the art that many changes and modifications may be made thereunto without the departing from the scope of the invention, which is defined by the following claims and their equivalents.

What is claimed:

1. A system for treating organ tissue, comprising:
a source of electrical energy;
a first electrode coupled to the energy source, the first electrode having a surface configured for electrically coupling with a surface of an organ; and
a second electrode coupled to the energy source, the second electrode having a tissue-piercing distal tip configured for piercing the organ such that the second electrode electrically couples with internal tissue of the organ.

2. The system of claim 1, further comprising a third electrode electrically coupled to one of the first and second electrodes.

3. The system of claim 2, further comprising a securing device for securing each of the first and the third electrodes relative to the organ surface.

4. The system of claim 3, the securing device comprising one or more elastic bands.

5. The system of claim 2, wherein the first and third electrodes are electrically coupled to form a first pole of a circuit, the second electrode forming a second pole of the circuit.

6. The system of claim 2, wherein the first and second electrodes are electrically coupled to form a first pole of a circuit, the third electrode forming a second pole of the circuit.

7. The system of claim 1, further comprising an elastic structure to which the first electrode is secured.

8. The system of claim 7, wherein the elastic structure is bendable from a first configuration to a second configuration upon application of force, and remains in its second configuration upon removal of the force.

9. The system of claim 1, wherein the first electrode surface is elastic and has a planar configuration.

10. A method of performing an organ tissue ablation procedure, comprising:
placing a first electrode at a first position on a surface of an organ;
piercing the organ with a second electrode to position the second electrode inside the organ; and
applying electrical energy through a circuit formed by the first and second electrodes to ablate a portion of the organ.

11. The method of claim 10, the organ comprising a liver.

12. The method of claim 10, further comprising placing a third electrode at a second position on the surface of the organ, wherein the first and third electrodes are electrically coupled to form a first pole of an electrical circuit, the second electrode forms a second pole of the circuit.

13. The method of claim 12, wherein the first, second, and third electrodes are positioned to lie approximately in a plane.

14. The method of claim 12, wherein the positions of the first and second electrodes define a first line, and wherein the positions of the second and third electrodes define a second line intersecting with the first line.

15. The method of claim 10, further comprising placing a third electrode at a second position on the surface of the organ, wherein the first and second electrodes are electrically coupled to form a first pole of an electrical circuit, and the third electrode forms a second pole of the circuit.

16. The method of claim 10, further comprising securing the first electrode relative to the surface of the organ using one or more elastic bands.

17. The method of claim 10, wherein the first electrode is bendable.

18. The method of claim 10, wherein the first electrode comprises a flexible envelope having a lumen for accommodating a part of the organ.

19. The method of claim 10, wherein the first electrode has a curvilinear planar configuration.

20. A system for treating organ tissue, comprising:
a source of electrical energy;
a first electrode coupled to the energy source and having a surface configured for electrically coupling with a surface of an organ at a first position; and
a second electrode coupled to the energy source and having a surface configured for electrically coupling with the surface of the organ at a second position.

21. The system of claim 20, further comprising a securing device for securing the first and the second electrodes relative to the surface of the organ.

22. The system of claim 21, wherein the securing device comprises one or more elastic bands.

23. The system of claim 20, further comprising an elastic structure to which the first electrode is secured.

24. The system of claim 23, wherein the elastic structure is bendable from a first configuration to a second configuration upon application of force, and remains in its second configuration upon removal of the force.

25. The system of claim 20, wherein the first electrode is elastic, and has a planar configuration.

26. A method of performing a liver ablation procedure, comprising:
placing a first electrode at a first position on a surface of a liver;
placing a second electrode at a second position on the surface the liver; and
applying electrical energy through an electrical circuit formed by the first and the second electrodes to ablate a portion of the liver.

27. The method of claim 26, wherein an entire cross section of the portion of the liver is ablated.

28. The method of claim 26, further comprising securing the first electrode relative to the liver surface.

29. The method of claim 26, wherein the first electrode is bendable.

30. The method of claim 26, wherein the first electrode has a curvilinear planar configuration.
